(19) United States
(12) Patent Application Publication
(10) Pub. No.: US 2025/0375583 A1
(43) Pub. Date: Dec. 11, 2025

(54) COUGH APPARATUS FOR ASSISTING A PATIENT IN COUGHING

(71) Applicant: Leowenstein Medical Technology S.A., Luxembourg (LU)

(72) Inventors: Lennart PAASCH, Hamburg (DE); Kris SCHWEIKERT, Hamburg (DE); Jan SCHATTNER, Pinneberg (DE)

(21) Appl. No.: 19/220,390

(22) Filed: May 28, 2025

(30) Foreign Application Priority Data
Jun. 5, 2024 (DE) 10 2024 115 615.5

(51) Int. Cl. A61M 16/00

(52) U.S. Cl. CPC A61M 16/0066; A61M 16/024; A61M 2016/0027; A61M 2016/0033; A61M 2205/3365; A61M 2206/20

(57) ABSTRACT

A cough apparatus comprises a blower with a suction opening, a blow-out opening, a drive motor, and a rotor coupled to the drive motor for conveying a gas from the suction opening to the blow-out opening depending on a motor speed; a pressure port; a patient port; a connection channel connecting the pressure port to the patient port; a coupling device switchable between an insufflation position and an exsufflation position; a pressure sensor designed to detect a pressure applied at the patient port and to generate a pressure signal indicating the detected pressure; and a control device for controlling an operation of the cough apparatus and being designed to carry out the method specified in the claims.

### CROSS-REFERENCE TO RELATED APPLICATIONS

The present application claims priority under 35 U.S.C. § 119 of German Patent Application No. 10 2024 115 615.5, filed Jun. 5, 2024, the entire disclosure of which is expressly incorporated by reference herein.

### BACKGROUND OF THE INVENTION

### 1. Field of the Invention

The invention relates to a cough apparatus for assisting a patient in coughing and to a ventilator having such a cough apparatus.

### 2. Discussion of Background Information

A cough apparatus, also called an insufflator-exsufflator, can be used to permit or facilitate the discharge of secretions from the airways and/or lungs of a patient with impaired respiratory muscles. For this purpose, the cough apparatus can alternately apply a positive pressure and a negative pressure to the airways and/or lungs via a suitable patient interface. This causes a cough or a series of coughs in a controlled manner. The efficiency of the discharge of secretions in a single cough depends, among other things, on the maximum of the volumetric flow during exsufflation, also known as the peak cough flow, or PCF for short. The PCF should be as high as possible in order to permit good discharge of secretions. At the same time, excessive pressure peaks during exsufflation should be avoided.

In view of the foregoing, it would be advantageous to have available a cough apparatus that permits particularly efficient discharge of secretions. It further would be advantageous to have available a ventilator with an improved cough apparatus.

### SUMMARY OF THE INVENTION

In a first aspect, the invention provides a cough apparatus for assisting a patient in coughing. The cough apparatus comprises: a blower with a suction opening, a blow-out opening, a drive motor, and a rotor coupled to the drive motor for conveying a gas from the suction opening to the blow-out opening depending on a motor speed of the drive motor; a pressure port; a patient port for connecting a patient interface; a connection channel fluidically connecting the pressure port to the patient port; a coupling device, which is switchable between an insufflation position and an exsufflation position and is configured to fluidically couple the pressure port to the blow-out opening in the insufflation position and to the suction opening in the exsufflation position; a throttle device, which is switchable between a starting position and a throttle position and is designed to narrow the connection channel when switching to the throttle position; a pressure sensor, which is designed to detect a pressure applied at the patient port and to generate a pressure signal indicating the detected pressure; and a control device for controlling an operation of the cough apparatus. The control device is configured to carry out the following insufflation method: switching the coupling device to the insufflation position (subsequently or in response to the switching of the coupling device to the insufflation position); receiving the pressure signal, wherein the pressure signal indicates an insufflation pressure applied at the patient port as the detected pressure; determining an insufflation pressure deviation between the insufflation pressure and an insufflation pressure setpoint; controlling the drive motor to reduce the insufflation pressure deviation; (when controlling the drive motor) switching the throttle device to the throttle position and holding the throttle device in the throttle position in order to cause an additional increase in the motor speed.

Such a cough apparatus permits a significant increase in the PCF without the need for a more powerful blower or for faster switching from the insufflation position to the exsufflation position. The throttling, especially toward the end of the insufflation, has the effect that a flow resistance that opposes the blower during the insufflation is additionally increased. Due to the pressure regulation, the motor speed increases accordingly. This makes it possible to provide a correspondingly increased volumetric flow in the subsequent exsufflation, especially if the increased motor speed is maintained at least until the time of switching to the exsufflation position.

The cough apparatus may comprise one or more electric or electrically controllable actuators for switching the coupling device and/or the throttle device. Such an actuator may be, for example, an electric motor, an electromagnet, a piezoelectric element or a combination of at least two of these examples.

For example, a “patient interface” can be understood to mean a mouth mask, a nose mask, a mouthpiece or a tube. For example, the patient port can be fluidically connectable to the patient interface via a tubing system.

The term “drive motor” can be understood in particular to mean an electric motor.

The coupling device and the throttle device may each be an electrically switchable valve. The switching positions of the coupling device and/or of the throttle device may be discrete switching positions. Alternatively, the switching positions of the coupling device and/or of the throttle device may be switching positions of a continuously adjustable valve.

The “starting position” can be understood to mean a position of the throttle device in which the connection channel is either not narrowed or is significantly less narrowed than in the throttle position.

The “throttle position” can be understood to mean a position of the throttle device in which the connection channel is either completely narrowed, i.e. closed in a gas-tight manner, or partially narrowed (for example by at least about 5% and/or at most about 95% relative to the starting position) so that gas can continue to flow through the connection channel. Depending on the embodiment, a switching speed at which the throttle device is moved from the starting position to the throttle position may be constant or may vary in time according to a predetermined profile.

“Narrowing” can generally be understood to mean an increase in a flow resistance that opposes the blower during operation of the cough apparatus. As mentioned above, the motor speed increases due to the pressure regulation in order to compensate for the pressure drop caused by the narrowing. In other words, the throttle device can generally be understood as a variable flow resistance.

The control device may comprise hardware and/or software components. For example, the control device may comprise a processor configured to execute a computer program for controlling the operation of the cough apparatus. In addition, the control device may comprise a memory and/or a data communication interface for wireless and/or wired data communication with peripheral devices. The control device may also be solely implemented as hardware, for example in the form of an ASIC component or FPGA component.

“Insufflation pressure” can be understood as an overpressure or positive pressure relative to the respective atmospheric pressure.

In a second aspect, the invention provides a ventilator for invasive and/or non-invasive ventilation of a patient. The ventilator comprises a cough apparatus as described above and below. In contrast to the cough apparatus, the ventilator may comprise additional ventilation functions for pressure-controlled and/or flow-controlled and/or volume-controlled ventilation of a patient.

Various embodiments of the invention are described below. These embodiments should not be understood as restricting the scope of the invention.

According to one embodiment, the throttle device may be held in the throttle position at least until the coupling device is switched to the exsufflation position. This permits the provision of a correspondingly increased volumetric flow at the beginning of exsufflation, which can improve the discharge of secretions.

According to one embodiment, the throttle device, in the insufflation method, may be switched to the throttle position and/or held in the throttle position taking into account a time profile of the insufflation pressure and/or a time profile of the motor speed. In this way, a suitable switching time for switching the throttle device between the starting position and the throttle position can be determined depending on the current operating conditions of the cough apparatus. Alternatively, the switching time may be fixed, for example depending on a (specified) duration of the insufflation method.

According to one embodiment, the throttle device may be switched to the throttle position if at least one of the following conditions is met:
the insufflation pressure, at least for a specified duration, is at least about 80%, preferably at least about 90% of the insufflation pressure setpoint;
the insufflation pressure setpoint is constant for at least a specified duration;
the motor speed, at least for a specified duration, is below a speed threshold value which, taking into account a maximum possible motor speed, is selected in such a way that a decrease in the insufflation pressure, which occurs when the throttle device is switched to the throttle position, can only just be compensated by increasing the motor speed beyond the speed threshold value.

According to one embodiment, the insufflation method may further comprise: preventing the throttle device from being switched to the throttle position if at least one of the following conditions is met:
the insufflation pressure setpoint is greater in magnitude than an exsufflation pressure setpoint, which is to be applied at the patient port in the case of an exsufflation;
the insufflation pressure setpoint is above a pressure threshold value which, taking into account a maximum possible insufflation pressure, is selected in such a way that a decrease in the insufflation pressure at the level of the pressure threshold value, which occurs when the throttle device is switched to the throttle position, can only just be compensated by increasing the motor speed;
a function for causing a targeted oscillation of the insufflation pressure is activated.

“Exsufflation pressure” can be understood to mean an under pressure or negative pressure relative to the respective atmospheric pressure.

According to one embodiment, the throttle device may be switchable to at least one additional throttle position and may be configured to narrow the connection channel when switching to the additional throttle position differently than when switching to the throttle position. In this case, the throttle device may pass the additional throttle position when switching between the starting position and the throttle position. It is possible that the connection channel is narrowed to a greater extent when the throttle is switched to the additional throttle position than when the throttle device is switched to the throttle position (or vice versa). In this way, the connection channel can be narrowed continuously and/or gradually, for example degressively and/or progressively. This permits better control of the narrowing of the connection channel compared to an embodiment with only one throttle position. Thus, undesired fluctuations in the insufflation pressure can be avoided when narrowing the connection channel.

According to one embodiment, the throttle device, when switching to the throttle position, may first be moved with a first switching speed (or in accordance with a specified first switching speed profile) from the starting position to the additional throttle position and then with a second switching speed different from the first switching speed (or in accordance with a specified second switching speed profile different from the first switching speed profile) from the additional throttle position to the throttle position. The first switching speed (on average) may be significantly greater than the second switching speed (or vice versa). In other words, it is possible that, when switching to the throttle position, the throttle device is first moved abruptly to the additional throttle position and then much more slowly to the throttle position. Such an embodiment with different switching speeds proved particularly favorable in tests.

According to one embodiment, the control device may be configured also to carry out the following exsufflation method, for example (immediately) after the insufflation method and/or (automatically) in response to conclusion of the insufflation method: switching the coupling device to the exsufflation position; switching the throttle device to the starting position; (if the coupling device has been switched to the exsufflation position) receiving the pressure signal, wherein the pressure signal indicates an exsufflation pressure applied at the patient port as the detected pressure; determining an exsufflation pressure deviation between the exsufflation pressure and an exsufflation pressure setpoint; controlling the drive motor to reduce the exsufflation pressure deviation. The switching of the coupling device may be carried out simultaneously with or (for example slightly) offset in time with respect to (for example before and/or after) the switching of the throttle device. The switching between the insufflation method and the exsufflation method may be done automatically and/or manually. The control unit may be expediently configured to switch alternately between the insufflation method and the exsufflation method several times in the operation of the cough apparatus (automatically).

According to one embodiment, the coupling device may further be switchable to at least one intermediate position and may be configured to fluidically couple the pressure port, in the intermediate position, both to the suction opening and to the blow-out opening. In this case, the exsufflation method may also comprise: switching the coupling device from the exsufflation position to the intermediate position in order to attenuate undesired fluctuations in the exsufflation pressure. The increased motor speed when switching to the exsufflation position may in some cases cause undesirable pressure fluctuations. In order to mitigate this effect without significantly influencing the pressure control, the coupling device may be briefly switched to the intermediate position after reaching the exsufflation position. The coupling device may also be switched several times between the exsufflation position and the intermediate position and/or between different intermediate positions, in order to attenuate the undesired pressure fluctuations. The coupling device may, for example, be configured to additionally aerate the connection channel in the intermediate position, so that an additional pressure equalization with an environment of the connection channel takes place.

According to one embodiment, the coupling device may be switched to the intermediate position and/or held in the intermediate position taking into account a time profile of the exsufflation pressure and/or a time profile of the motor speed. In this way, a suitable switching time for switching the coupling device between the exsufflation position and the intermediate position may be determined depending on the current operating conditions of the cough apparatus. Alternatively, the switching time may be fixed, for example depending on a (specified) duration of the exsufflation method.

According to one embodiment, an amplitude of the pressure signal and/or of a speed signal indicating the motor speed may be compared in several successive time steps with a predetermined tolerance range, between the limits of which the amplitude may fluctuate, wherein the coupling device may be switched between the exsufflation position and the intermediate position depending on a deviation of the amplitude from at least one of the limits. For example, the tolerance range may define a range of fluctuation of the order of plus/minus about 20%, plus/minus about 10%, plus/minus about 5%, or plus/minus about 1% around a desired average value of the amplitude. If, for example, it is detected that the amplitude is in the tolerance range for at least a specified duration, the coupling device may be switched back to the exsufflation position and held in the exsufflation position until the end of the exsufflation method.

Alternatively, the coupling device may be held in the intermediate position in a fixed time window.

According to one embodiment, the throttle device may further be switchable between a first oscillation position and a second oscillation position and may be configured to narrow the connection channel when switching from the first oscillation position to the second oscillation position. In this case, the following step may also be carried out in the insufflation and/or exsufflation method: alternately switching the throttle device between the first oscillation position and the second oscillation position in order to cause a targeted oscillation of the insufflation or exsufflation pressure. This may further improve the discharge of secretions. The alternating switching may take place after the (first) switching of the throttle device to the starting position and/or taking into account a time profile of the insufflation or exsufflation pressure and/or a time profile of the motor speed. For example, the alternating switching may take place only when it is detected that the (measured) insufflation or exsufflation pressure or the insufflation or exsufflation pressure setpoint is constant at least for a specified duration. “Oscillation position” can be understood, hereinabove and hereinbelow, as a fixed switching position of the throttle device or as a switching position variable during the operation of the cough apparatus. The switching position may, for example, be variable depending on the time profile of the pressure signal and/or of a speed signal indicating the motor speed and/or depending on a specified oscillation profile.

According to one embodiment, the first oscillation position may be the starting position. Alternatively, the first oscillation position may deviate from the starting position and, for example, may be a position between the starting position and the throttle position.

According to one embodiment, the second oscillation position may be the throttle position. Alternatively, the second oscillation position may deviate from the throttle position. In other words, the throttle device may be configured to narrow the connection channel when switching to the second oscillation position differently than when switching to the throttle position (and/or differently from when switching to the aforementioned additional throttle position). Thus, it is possible that the connection channel is narrowed to a greater extent when the throttle device is switched to the second oscillation position than when the throttle device is switched to the throttle position (or vice versa). It is possible that the throttle device passes the throttle position (and, if necessary, the aforementioned additional throttle position) when switching between the first oscillation position and the second oscillation position. This allows, for example, the connection channel to be continuously and/or gradually narrowed or expanded, for example degressively and/or progressively, in order to cause the targeted oscillation.

If a targeted oscillation of the insufflation pressure is to be effected in the insufflation method, it is advantageous to prevent the switching of the throttle device to the throttle position for the purpose of increasing the motor speed further.

According to one embodiment, the cough apparatus may further comprise a flow sensor, which is designed to detect a volumetric flow through the connection channel and to generate a flow signal indicating the detected volumetric flow. In this case, the control device may be configured to further evaluate the flow signal for switching the coupling device and/or the throttle device and/or for controlling the drive motor. The flow sensor may, for example, be designed to detect a volumetric flow through a portion of the connection channel located between the throttle device and the patient port. This permits more precise control of the operation of the cough apparatus compared to an embodiment in which the volumetric flow is not taken into account.

According to one embodiment, the exsufflation method may further comprise: receiving the flow signal, wherein the flow signal indicates an exsufflated volumetric flow as the detected volumetric flow, and evaluating the flow signal in order to detect a local maximum in the time profile of the detected volumetric flow. In this case, the alternating switching to cause the targeted oscillation may be done in response to the detection of the local maximum, for example the peak cough flow.

According to one embodiment, the throttle device may comprise an actuating element, which is rotatably mounted about an axis of rotation between the starting position and the throttle position, and an electric or electrically controllable actuator for rotating the actuating element. The actuating element may comprise a wall portion for reducing a flow cross section of the connection channel and may be configured in such a way that the wall portion protrudes further into the connection channel in the throttle position than in the starting position.

According to one embodiment, the cough apparatus may further comprise an aeration duct leading into the connection channel in order to permit a pressure equalization between the interior of the connection channel and an external environment of the connection channel. In this case, the throttle device may be designed to close the aeration duct in the starting position and to release it in the throttle position. This can be understood to mean that the aeration duct is narrowed to a greater extent when the throttle is switched to the starting position than when the throttle device is switched to the throttle position. For example, in the starting position, the aeration duct can be either completely narrowed, i.e. closed in a gas-tight manner, or partially narrowed (for example by at least about 5% and/or at most about 95% relative to the throttle position) so that gas can continue to flow through the aeration duct. This allows a slight internal leakage to be generated when the throttle device is switched to the throttle position, which may have a favorable effect on the pressure profile.

In addition, the throttle device may be configured to close the aeration duct in the first oscillation position and/or to release it in the second oscillation position.

### BRIEF DESCRIPTION OF THE DRAWINGS

Embodiments of the invention are described hereinafter with reference to the accompanying drawings. Neither the description nor the drawings should be understood as restricting the scope of the invention. In the drawings:

FIG. 1 shows a cough apparatus according to an embodiment of the invention with a coupling device in an insufflation position and a throttle device in a starting position.

FIG. 2 shows the cough apparatus from FIG. 1, wherein the throttle device is in a first throttle position.

FIG. 3 shows the cough apparatus from FIG. 1, wherein the throttle device is in a second throttle position.

FIG. 4 shows the cough apparatus from FIG. 1, wherein the coupling device is in an exsufflation position.

FIG. 5 shows the cough apparatus from FIG. 4, wherein the coupling device is in an intermediate position.

FIG. 6 shows the cough apparatus from FIG. 4, wherein the throttle device is in an oscillation position.

FIG. 7 shows a diagram illustrating a time profile of a pressure and of a volumetric flow during operation of a cough apparatus according to an embodiment of the invention.

The drawing are purely schematic and not true to scale. If identical reference signs are used in different drawings, then these reference signs designate identical or identically acting features.

### DETAILED DESCRIPTION OF EXEMPLARY EMBODIMENTS

The particulars shown herein are by way of example and for purposes of illustrative discussion of the embodiments of the present invention only and are presented in the cause of providing what is believed to be the most useful and readily understood description of the principles and conceptual aspects of the present invention. In this regard, no attempt is made to show details of the present invention in more detail than is necessary for the fundamental understanding of the present invention, the description in combination with the drawings making apparent to those of skill in the art how the several forms of the present invention may be embodied in practice.

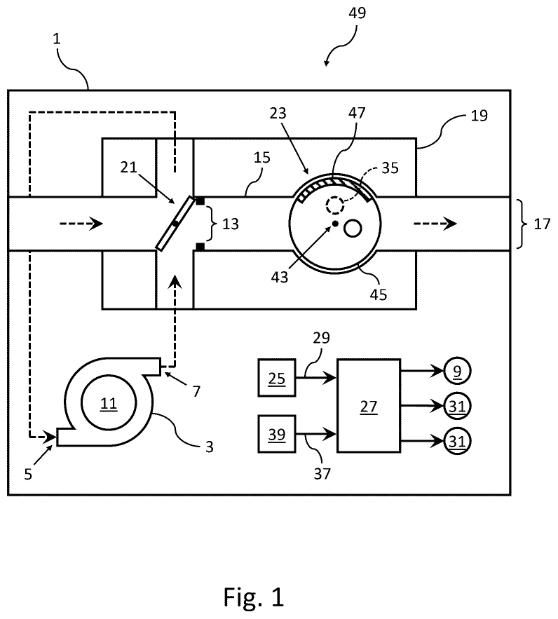

FIG. 1 shows a cough apparatus 1 for assisting a patient in coughing. The cough apparatus 1 comprises a blower 3 having a suction opening 5, a blow-out opening 7, a drive motor 9, and a rotor 11 coupled to the drive motor 9 for conveying a gas from the suction opening 5 to the blow-out opening 7 depending on a motor speed of the drive motor 9. A direction of flow of the gas is indicated by dashed arrows.

Furthermore, the cough apparatus 1 comprises a pressure port 13 and a patient port 17 fluidically connected to the pressure port 13 via a connection channel 15, for connecting a patient interface, for example, a mask, a mouthpiece, an endotracheal cannula or a tracheostomy cannula. The two ports 13, 15 can be formed, for example, as ports of a pneumatic unit 19. The pneumatic unit 19 can be designed, for example, as a valve housing or valve block.

In addition, the cough apparatus 1 comprises a coupling device 21, a throttle device 23, a pressure sensor 25, and a control device 27 for controlling an operation of the cough apparatus 1.

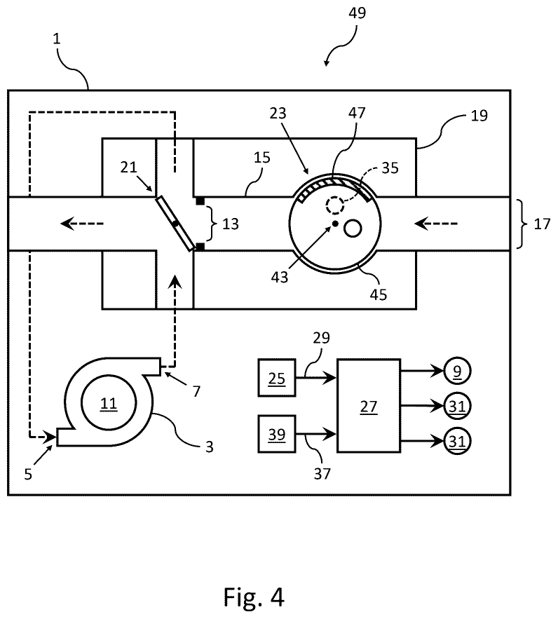

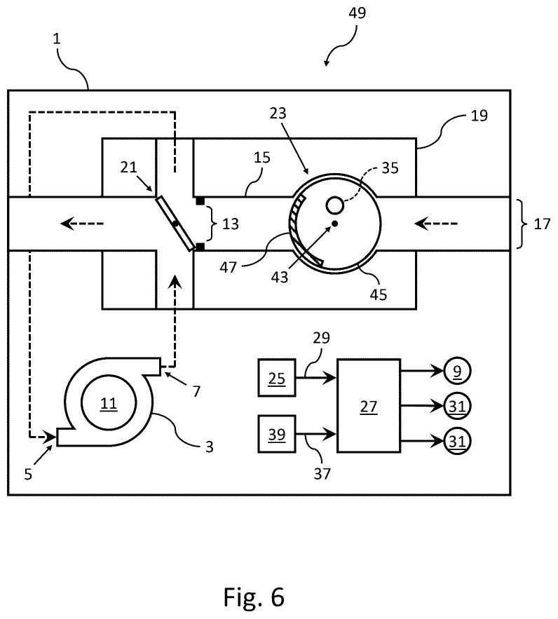

The coupling device 21, here in the form of a flap valve, is switchable between an insufflation position and an exsufflation position and is designed to fluidically connect the pressure port 13 to the blow-out opening 7 in the insufflation position (see FIG. 1 to FIG. 3) and to the suction opening 5 in the exsufflation position (see FIG. 4 and FIG. 6).

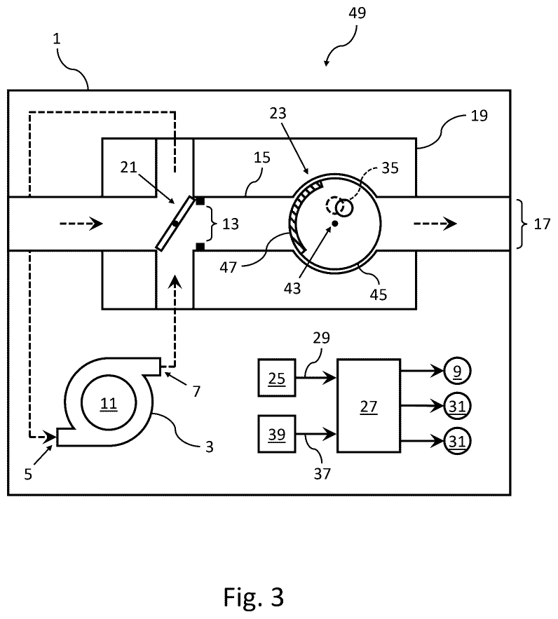

The throttle device 23 is switchable between a starting position (see FIG. 1 and FIG. 4) and a throttle position (see FIG. 3) and is designed to specifically narrow the connection channel 15 when switching from the starting position to the throttle position.

The pressure sensor 25 is designed to detect a pressure applied at the patient port 17 and to generate a pressure signal 29 indicating the detected pressure.

The control device 27 is designed to carry out the following insufflation method: switching the coupling device 21 to the insufflation position; receiving the pressure signal 29, wherein the pressure signal 29 indicates an insufflation pressure applied at the patient port 17 as the detected pressure; determining an insufflation pressure deviation between the insufflation pressure and an insufflation pressure setpoint, which has been set manually for example; controlling the drive motor 9 to reduce the insufflation pressure deviation; when controlling the drive motor 9: switching the throttle device 23 to the throttle position and holding the throttle device 23 in the throttle position in order to cause an additional increase in the motor speed.

For example, the throttle device 23 can be held in the throttle position until the coupling device 21 is switched to the exsufflation position in order to initiate an exsufflation. It is also conceivable that the throttle device 23 is held in the throttle position briefly until beyond the time of switching to the exsufflation position. Alternatively, it is possible that the throttle device 23 is already switched back to the starting position shortly before the time of the switching to the exsufflation position.

This permits a significant increase in PCF without the need for a more powerful blower or for faster switching from the insufflation position to the exsufflation position. The throttling, especially toward the end of the insufflation, has the effect that a flow resistance that opposes the blower during the insufflation is additionally increased. Due to the pressure regulation, the motor speed increases accordingly. This makes it possible to provide a correspondingly increased volumetric flow in the subsequent exsufflation.

It is possible that the coupling device 21 and the throttle device 23 each comprise an electric or electrically controllable actuator 31 for switching between the respective switching positions. Such an actuator 31 can be, for example, an electric motor, an electromagnet, a piezoelectric element or a combination of at least two of these examples.

In addition, the control device 27 can be designed to carry out the following exsufflation method following the insufflation method: switching the coupling device 21 to the exsufflation position; switching the throttle device 23 back to the starting position, for example simultaneously with the switching of the coupling device 21 to the exsufflation position; receiving the pressure signal 29, wherein the pressure signal 29 indicates an exsufflation pressure applied at the patient port 17 as the detected pressure; determining an exsufflation pressure deviation between the exsufflation pressure and an exsufflation pressure setpoint, which has also been set manually for example; controlling the drive motor 9 to reduce the exsufflation pressure deviation.

The switching between the insufflation method and the exsufflation method can be done manually and/or automatically.

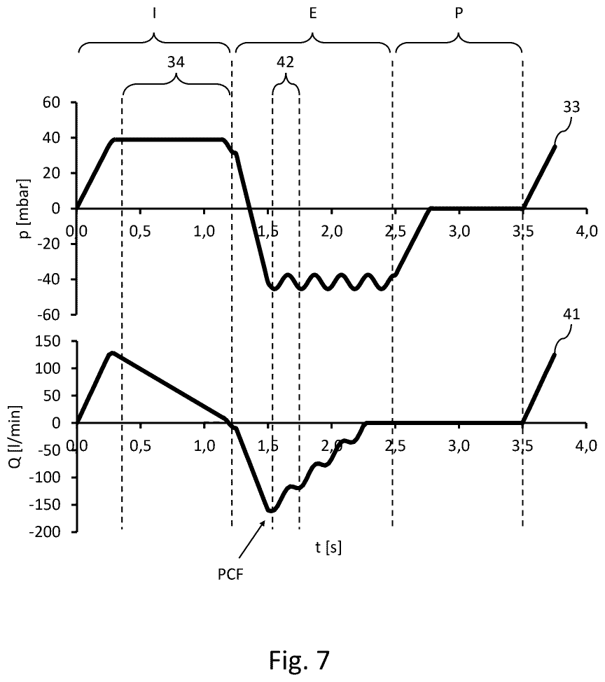

For example, the control device 27 can be designed to switch the cough apparatus 1 to a pause position (see the section “P” in FIG. 7) between two cough cycles, wherein the cough apparatus 1 is designed such that, in the pause position, a respective atmospheric pressure is applied at the patient port 17.

It is expedient if the throttle device 23, in the insufflation method, is switched to the throttle position and/or held in the throttle position taking into account a time profile 33 (see FIG. 7) of the insufflation pressure and/or a time profile of the motor speed. This makes it possible to control the throttle device 23 depending on the current operating conditions of the cough apparatus 1. However, the throttle device 23 can also be switched at a fixed switching time. In FIG. 7, a possible throttling time window 34, in which the throttling device 23 is held in the throttling position, is marked as an example.

The control device 27 can be designed to switch the throttle device 23 to the throttle position depending on whether certain switching conditions are met.

The switching conditions can comprise, for example, the following necessary conditions:
the insufflation pressure setpoint is at most as high as the exsufflation pressure setpoint (otherwise the drive motor 9 is operated in the insufflation method at a higher motor speed than in the exsufflation method anyway);
the insufflation pressure setpoint is at most as high as a predetermined pressure threshold value which, taking into account a maximum possible insufflation pressure, is selected in such a way that a decrease in the insufflation pressure at the level of the pressure threshold value, which occurs when the throttle device 23 is switched to the throttle position, can only just be compensated by increasing the motor speed (if the insufflation pressure setpoint exceeds the pressure threshold value, then in some circumstances there is no longer a sufficient speed reserve);
any function for causing a targeted oscillation of the insufflation pressure is deactivated.

Depending on the embodiment of the cough apparatus 1, in particular of the blower 3, the pressure threshold value can be, for example, between 40 hPa and 70 hPa, in particular between 50 hPa and 60 hPa.

If at least one of the necessary conditions does not apply, the control device 27 prevents the switching of the throttle device 23 to the throttle position during insufflation.

In addition, the switching conditions can comprise, for example, the following sufficient conditions:
the (measured) insufflation pressure, at least for a specified duration, is at least about 80%, preferably at least about 90% of the insufflation pressure setpoint;
the insufflation pressure setpoint is constant at least for a specified duration (i.e. the insufflation pressure setpoint has reached a specified plateau phase);
the motor speed, at least for a specified duration, is below a specified speed threshold value which, taking into account a maximum possible motor speed, is selected in such a way that a decrease in the insufflation pressure, which occurs when the throttle device 23 is switched to the throttle position, can only just be compensated by increasing the motor speed beyond the speed threshold value.

If each of the sufficient conditions applies, the control device 27 switches the throttle device 23 to the throttle position during insufflation.

Depending on the embodiment of the cough apparatus 1, in particular of the blower 3, the speed threshold value can be, for example, from about 20,000 to about 60,000, in particular from about 35,000 to about 45,000, preferably about 42,000 revolutions per minute.

The specified duration can be, for example, from about 0.1 second to about 1 second, preferably from about 0.4 second to about 0.7 second, in particular about 0.5 second.

The verification as to whether the above conditions are met can be carried out in several successive time steps, for example from about 50 to about 200 times per second, in particular from about 90 to about 110 times per second.

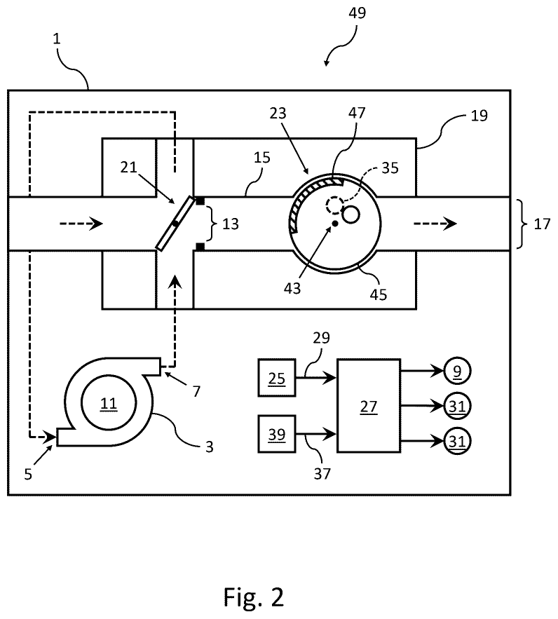

As is shown in FIG. 2, the throttle device 23 is switchable to at least one additional throttle position located between the starting position and the throttle position and can be designed to narrow the connection channel 15 when switching to the additional throttle position differently, for example more strongly, than when switching to the throttle position. The insufflation method commences with the starting position. If the aforementioned conditions are met, the throttle device 23 is, for example, initially switched abruptly to the additional throttle position, in which a flow resistance opposing the blower 3 is significantly increased in comparison to the starting position. The throttle device 23 is then moved from the additional throttle position to the throttle position at a reduced actuating speed. For example, in the starting position (and optionally in the additional throttle position), the throttle device 23 can be designed to close in an airtight manner an aeration duct 35 leading into the connection channel 15 and, in the throttle position, to partially release said aeration duct, such that an additional pressure equalization with an environment of the connection channel 15 takes place in the throttle position. This can have a favorable effect on the pressure gradient.

In addition, the throttle device 23 can be switchable between a first oscillation position and a second oscillation position and can be designed to narrow the connection channel 15 when switching from the first oscillation position to the second oscillation position. In this case, it is possible that the throttle device 23 in the insufflation and/or exsufflation method is switched several times alternately between the first oscillation position and the second oscillation position in order to cause a targeted oscillation of the insufflation and/or exsufflation pressure.

In this example, the first oscillation position matches the starting position, while the second oscillation position (see FIG. 6) is a position which deviates from the throttle position or from the various throttle positions and in which the throttle device 23 completely releases the aeration duct 35, in contrast to the throttle position or to the throttle positions.

It is possible that the control device 27, in addition to the pressure signal 29, receives a flow signal 37 from a flow sensor 39 for detecting a volumetric flow through a portion of the connection channel 15 located between the throttle device 23 and the patient port 17. In this case, the alternating switching between the first oscillation position and the second oscillation position in the exsufflation method can only take place, for example, when it is detected from the flow signal 37 that an exsufflated volumetric flow has reached a local maximum, also called PCF. FIG. 7 shows an example of a time profile 41 of the volumetric flow in the operation of the cough apparatus 1.

The control device 27 can also be designed to evaluate the flow signal 37 for controlling the drive motor 9 and/or the coupling device 21.

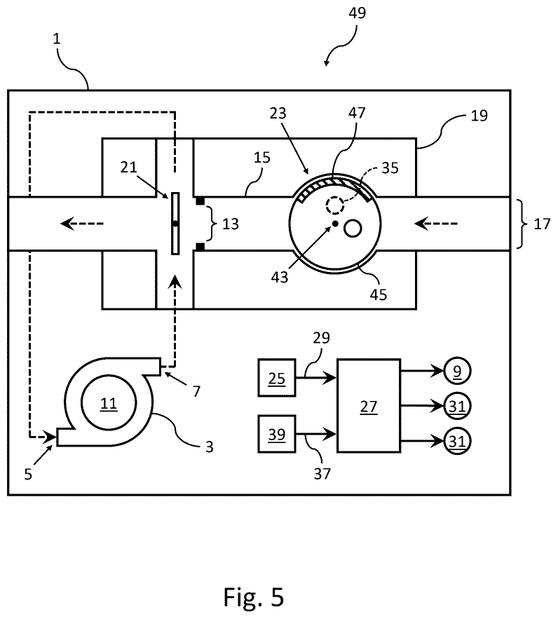

In order to dampen excessive pressure peaks at the beginning of the exsufflation process, the coupling device 21 can also be switched to at least one intermediate position (see FIG. 5) and be designed to fluidically couple the pressure port 13 in the intermediate position both to the suction opening 5 and to the blow-out opening 7. In FIG. 7, a possible damping time window 42 is marked as an example, in which the coupling device 21 is held in the intermediate position or is alternately switched between the exsufflation position and the intermediate position (and/or between different intermediate positions).

For example, the control device 27 can be designed to switch the coupling device 21 between the exsufflation position and the intermediate position (and/or between the various intermediate positions), taking into account a time profile 33 of the exsufflation pressure and/or a time profile of the motor speed. For this purpose, for example, an amplitude of the pressure signal 29 and/or of a speed signal indicating the motor speed can be compared, in several successive time steps, with a predetermined tolerance range, between whose limits the amplitude may fluctuate. Accordingly, the coupling device 21 can be switched between the exsufflation position and the intermediate position (and/or between the various intermediate positions) depending on a deviation of the amplitude from at least one of the limits.

The throttle device 23 can, for example, comprise an actuating element 45 which is rotatable about an axis of rotation 43 between the starting position and an end position by means of the respective actuator 31 and which has a wall portion 47 for reducing the flow cross section of the connection channel 15. In this example, the end position is the second oscillation position, wherein the actuating element 45 passes the throttle position (or the various throttle positions) upon rotation between the starting position (which here corresponds to the first oscillation position) and the end position. In this case, the throttle device 23 can be designed such that the wall portion 47 protrudes further into the connection channel 15 in the end position and/or in each throttle position than it does in the starting position.

The cough apparatus 1 can be designed as a stand-alone apparatus or as a component of a ventilator 49 for invasive and/or non-invasive ventilation of the patient.

Finally, it will be noted that terms such as “have”, “comprise”, “include”, “with” etc. do not exclude other elements or steps, and indefinite articles such as “a” or “an” do not exclude a plurality.

Furthermore, it is pointed out that features or steps described with reference to one of the embodiments above can also be used in combination with features or steps described with reference to other embodiments from among the embodiments above.

Reference signs in the claims should not be understood as restricting the scope of the subject matter defined by the claims.

List of reference numerals

| | |
|---|---|
| 1 | cough apparatus |
| 3 | blower |
| 5 | suction opening |
| 7 | blow-out opening |
| 9 | drive motor |
| 11 | rotor |
| 13 | pressure port |
| 15 | connection channel |
| 17 | patient port |
| 19 | pneumatic unit |
| 21 | coupling device |
| 23 | throttle device |
| 25 | pressure sensor |
| 27 | control device |
| 29 | pressure signal |
| 31 | actuator |
| 33 | time profile of a pressure applied at |
| | the patient port |
| 34 | throttling time window |
| 35 | aeration duct |
| 37 | flow signal |
| 39 | flow sensor |
| 41 | time profile of a volumetric flow |
| | through the connection channel |
| 42 | attenuation time window |
| 43 | axis of rotation |
| 45 | actuating element |
| 47 | wall portion |
| 49 | ventilator |
| p | pressure |
| t | time |
| E | exsufflation |
| I | insufflation |
| P | pause |
| PCF | peak cough flow |
| Q | volumetric flow |

What is claimed is:

1. A cough apparatus for assisting a patient in coughing, wherein the cough apparatus comprises:
a blower with a suction opening, a blow-out opening, a drive motor, and a rotor coupled to the drive motor for conveying a gas from the suction opening to the blow-out opening depending on a motor speed of the drive motor;
a pressure port;
a patient port for connecting a patient interface;
a connection channel fluidically connecting the pressure port to the patient port;
a coupling device switchable between an insufflation position and an exsufflation position and being configured to fluidically couple the pressure port to the blow-out opening in an insufflation position and to the suction opening in an exsufflation position;
a throttle device switchable between a starting position and a throttle position and being configured to narrow the connection channel when switching to the throttle position;
a pressure sensor configured to detect a pressure (p) applied at the patient port and to generate a pressure signal indicating the detected pressure (p);
a control device for controlling an operation of the cough apparatus and being configured to carry out the following insufflation method:
switching the coupling device to the insufflation position;
receiving the pressure signal, the pressure signal indicating an insufflation pressure (p) applied at the patient port as the detected pressure (p);
determining an insufflation pressure deviation between the insufflation pressure (p) and an insufflation pressure setpoint;
controlling the drive motor to reduce the insufflation pressure deviation;
switching the throttle device to the throttle position and holding the throttle device in the throttle position in order to cause an additional increase in the motor speed.

1. The cough apparatus of claim 1, wherein the throttle device is held in the throttle position at least until the coupling device is switched to the exsufflation position; and/or wherein the throttle device is switched to the throttle position and/or held in the throttle position taking into account a time profile of the insufflation pressure (p) and/or a time profile of the motor speed.

3. The cough apparatus of claim 1, wherein the throttle device is switched to the throttle position when at least one of the following conditions is met:
the insufflation pressure (p), for at least a specified duration, is at least 80% of the insufflation pressure setpoint;
the insufflation pressure setpoint is constant for at least a specified duration;
the motor speed, for at least a specified duration, is below a speed threshold value which, taking into account a maximum possible motor speed, is selected in such a way that a decrease in the insufflation pressure (p), which occurs when the throttle device (23) is switched to the throttle position, can only just be compensated by increasing the motor speed beyond the speed threshold value.

4. The cough apparatus of claim 1, wherein the insufflation method further comprises preventing the throttle device from being switched to the throttle position if at least one of the following conditions is met:
the insufflation pressure setpoint is greater in magnitude than an exsufflation pressure setpoint, which is intended to be present at the patient port in the case of an exsufflation;
the insufflation pressure setpoint is above a pressure threshold value which, taking into account a maximum possible insufflation pressure, is selected in such a way that a decrease in the insufflation pressure (p) at the level of the pressure threshold value, which occurs when the throttle device is switched to the throttle position, can only just be compensated by increasing the motor speed;
a function for causing a targeted oscillation of the insufflation pressure is activated.

5. The cough apparatus of claim 1, wherein the throttle device is switchable to at least one additional throttle position and is configured to narrow the connection channel when switching to the additional throttle position differently than when switching to the throttle position, wherein the throttle device passes the additional throttle position when switching between the starting position and the throttle position.

6. The cough apparatus of claim 5, wherein the throttle device, when switching to the throttle position, is first moved with a first switching speed from a starting position to an additional throttle position and then with a second switching speed from the additional throttle position to the throttle position.

7. The cough apparatus of claim 1, wherein the control device is configured to further carry out the following exsufflation method:
switching the coupling device to the exsufflation position;
switching the throttle device to the starting position;
receiving the pressure signal, the pressure signal indicating an exsufflation pressure (p) applied at the patient port as the detected pressure (p);
determining an exsufflation pressure deviation between the exsufflation pressure (p) and an exsufflation pressure setpoint;
controlling the drive motor to reduce the exsufflation pressure deviation.

8. The cough apparatus of claim 7, wherein the coupling device is further switchable to at least one intermediate position and is configured to fluidically couple the pressure port, in the intermediate position, both to the suction opening and to the blow-out opening, and wherein the exsufflation method further comprises:
switching the coupling device from the exsufflation position to the intermediate position in order to attenuate undesired fluctuations in the exsufflation pressure (p).

9. The cough apparatus of claim 8, wherein the coupling device is switched to the intermediate position and/or held in the intermediate position taking into account a time profile of the exsufflation pressure (p) and/or a time profile of the motor speed.

10. The cough apparatus of claim 7, wherein the throttle device is further switchable between a first oscillation position and a second oscillation position and is configured to narrow the connection channel when switching from the first oscillation position to the second oscillation position and wherein the exsufflation method further comprises:
alternately switching the throttle device between the first oscillation position and the second oscillation position in order to cause a targeted oscillation of the exsufflation pressure (p).

11. The cough apparatus of claim 1, wherein the cough apparatus further comprises a flow sensor, which is configured to detect a volumetric flow (Q) through the connection channel and to generate a flow signal indicating the detected volumetric flow (Q); the control device being configured to further evaluate the flow signal for switching the coupling device and/or the throttle device and/or for controlling the drive motor.

12. The cough apparatus of claim 11, wherein the exsufflation method further comprises receiving the flow signal, the flow signal indicating an exsufflated volumetric flow (Q) as the detected volumetric flow (Q) and wherein the alternating switching to cause the targeted oscillation takes place only when the exsufflated volumetric flow (Q) reaches a local maximum (PCF).

13. The cough apparatus of claim 1, wherein the throttle device comprises an actuating element rotatably mounted about an axis of rotation between a starting position and the throttle position, and an electric actuator for rotating an actuating element, the actuating element comprising a wall portion for reducing a flow cross section of the connection channel and being configured in such a way that the wall portion protrudes further into the connection channel in a throttle position than in a starting position.

14. The cough apparatus of claim 1, wherein the apparatus further comprises an aeration duct leading into the connection channel in order to permit a pressure equalization with an environment of the connection channel. the throttle device being configured to close the aeration duct in a starting position and to release it in a throttle position.

15. A ventilator for invasive and/or non-invasive ventilation of a patient, wherein the ventilator comprises the cough apparatus of claim 1.
